(19) United States
(12) Patent Application Publication
(10) Pub. No.: US 2008/0085311 A1
(43) Pub. Date: Apr. 10, 2008

(54) ANTIHISTAMINE-DECONGESTANT COMBINATIONS

(76) Inventors: Sanjay Shekhranand Tripathi, Nagpur (IN); Ravi Kumar Namballa, Hyderabad (IN); Vasanth Kumar Shetty, Mumbai (IN); Raghupathi Kandarapu, Hyderabad (IN); Srinivas Irukulla, Hyderabad (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/867,076

(22) Filed: Oct. 4, 2007

Related U.S. Application Data
(60) Provisional application No. 60/884,936, filed on Jan. 15, 2007.

(30) Foreign Application Priority Data
Oct. 5, 2006 (IN) 1846/CHE/2006

(51) Int. Cl. A61K 9/36; A61K 31/445; A61K 9/14; A61P 37/08; A61K 9/16

(52) U.S. Cl. 424/465; 424/489; 424/490; 424/495; 514/317

(57) ABSTRACT

Multi-particulate pharmaceutical compositions comprising an antihistamine for immediate release and a decongestant for modified release.

The present invention relates to pharmaceutical compositions for oral administration comprising an antihistamine and a decongestant, processes for preparing such compositions, and their methods of use. More particularly, the present invention relates to multi-particulate pharmaceutical compositions comprising an antihistamine for immediate release and a decongestant for modified release.

Antihistaminic and decongestant act by different mechanisms to treat allergic reactions. Decongestants constrict vessels in the nasal mucus membranes and thereby decrease tissue swelling and nasal congestion. Antihistamines block the binding of histamines to the histamine receptors by preoccupying the histaminic receptors. Consequently they are effective only if given prior to histamine release, since once histamine is released and binds to the receptors the binding cannot be reversed.

Combining decongestants and antihistamines utilizes both mechanistic approaches, and has been shown to offer more complete relief of rhinitis symptoms than therapy with either component alone. A commercially available product ALLEGRA-D® 24 HOUR Extended-Release Tablets (manufactured by Sanofi-Aventis, US) for oral administration contain 180 mg of fexofenadine hydrochloride for immediate release and 240 mg of pseudoephedrine hydrochloride for extended release. ALLEGRA-D® 24 HOUR extended-release tablets are indicated for the relief of symptoms associated with seasonal allergic rhinitis in adults and children 12 years of age and older.

Fexofenadine hydrochloride is a selective peripheral histamine H1-receptor antagonist having a chemical name (±)-4-[1-hydroxy-4-[4-(hydroxydiphenyl methyl)-1-piperidinyl]-butyl]-α,α-dimethyl benzeneacetic acid hydrochloride and is structurally represented by Formula I.

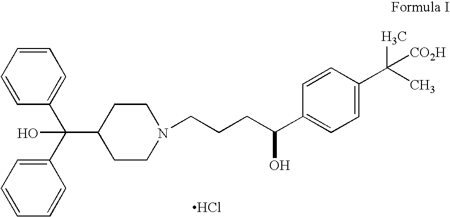

The molecular weight of fexofenadine hydrochloride is 538.1 and the empirical formula is C₃₂H₃₉NO₄.HCl. It is a white to off-white crystalline powder. It is freely soluble in methanol and ethanol, slightly soluble in chloroform and water, and insoluble in hexane. Fexofenadine hydrochloride is a racemate and exists as a zwitterion in aqueous media at physiological pH.

Pseudoephedrine hydrochloride is an adrenergic (vasoconstrictor) agent with the chemical name [S-(R*,R*)]-α-[1-(methylamino)ethyl]-benzenemethanol hydrochloride and is structurally represented by formula II. Pseudoephedrine hydrochloride is an orally active sympathomimetic amine and exerts a decongestant action on the nasal mucosa.

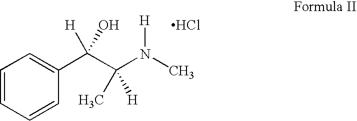

The molecular weight of pseudoephedrine hydrochloride is 201.7 and the molecular formula is C₁₀H₁₅NO.HCl. It occurs as fine, white to off-white crystals or powder, having a faint characteristic odor. It is very soluble in water, freely soluble in alcohol, and sparingly soluble in chloroform.

U.S. Pat. Nos. 6,613,357, 6,004,582 and RE39,069 disclose osmotic devices comprising pseudoephedrine and an H1 antagonist for oral delivery.

U.S. Pat. Nos. 6,994,871 and 6,039,974 describe once a day antihistamine and decongestant formulations.

U.S. Pat. No. 5,807,579 and U.S. Patent Application Publication No. 2006/0182800 disclose pharmaceutical compositions for antihistaminic-decongestant combinations.

Most of the formulation technologies adopted for formulating antihistaminic-decongestant combinations are based on an osmotic delivery concept, core and coating concept, or bilayer technology. It has been surprisingly found that the pharmaceutical compositions of the present invention comprising multi-particulate systems exhibit desired in vitro dissolution and in vivo absorption profiles.

Thus, the present invention provides for commercially viable alternative pharmaceutical compositions of antihistaminic-decongestant combinations for oral administration.

This and other such needs are addressed by the present invention.

### SUMMARY OF THE INVENTION

An aspect of the present invention provides multi-particulate pharmaceutical compositions comprising an antihistamine for immediate release and a decongestant for modified release.

Another aspect of the present invention provides multi-particulate pharmaceutical compositions wherein an antihistamine and a decongestant are separated by a release-controlling film coating, which provides modified release of said decongestant.

Still further aspect of the present invention provides multi-particulate pharmaceutical compositions wherein a decongestant, optionally with a binder or adherent and a release-controlling polymer, is coated onto an inert core, and the decongestant-coated inert core is further coated with a release-controlling polymer for modified release.

In one embodiment of the present invention, cores comprise a decongestant and pharmaceutically acceptable excipients, which are coated with a release-controlling film coating that provides modified release of the decongestant.

In another embodiment of the present invention, cores comprise a decongestant and a release-controlling polymer for providing modified release, and the modified release cores are optionally coated with a release-controlling or conventional film coating.

In one embodiment, cores providing modified release of the decongestant are granulated using a solution or dispersion comprising an antihistamine for immediate release.

In another embodiment, multi-particulate systems of the present invention are compressed into tablets without substantially damaging the modified release coating.

In another embodiment, multi-particulate systems of the present invention are formulated as capsules.

An embodiment of the invention provides a pharmaceutical composition comprising a plurality of formulated particles providing a modified release of pseudoephedrine or a salt thereof into aqueous fluids, granulated with a granulating composition containing fexofenadine or a salt thereof.

Another embodiment of the invention provides a pharmaceutical composition comprising a plurality of cellulose particles having a coating comprising pseudoephedrine or a salt thereof and having an outer coating comprising a polymer and providing a modified release of pseudoephedrine or a salt thereof into aqueous fluids, granulated with a granulating composition containing fexofenadine or a salt thereof.

A further embodiment of the invention provides a pharmaceutical composition comprising:

a) a plurality of cellulose particles having a coating comprising pseudoephedrine hydrochloride, an outer coating comprising ethyl cellulose, and providing a modified release of pseudoephedrine or a salt thereof into aqueous fluids, granulated with a granulating composition containing fexofenadine hydrochloride; and

b) one or more pharmaceutical excipients; compressed into a tablet.

### DETAILED DESCRIPTION

The present invention relates to multi-particulate pharmaceutical compositions comprising an antihistamine for immediate release and a decongestant for modified release. Pharmaceutically acceptable salts in the form of inorganic or organic acid or base addition salts for fexofenadine and pseudoephedrine such as inorganic acids are, for example, hydrochloric, hydrobromic, sulfuric, and phosphoric acids.

In one embodiment of present invention, an antihistamine comprises a H1-receptor antagonist, for example, fexofenadine or its pharmaceutically acceptable salts, solvates, polymorphs, enantiomers, single isomer, or mixtures thereof. Pharmaceutically acceptable salts of fexofenadine include, withut limitation thereto, the hydrochloride, hydrobromide, acetate, mesylate and sulphate salts.

In another embodiment of present invention, a decongestant comprises a sympathomimetic amine, for example, pseudoephedrine or its pharmaceutically acceptable salts, solvates, polymorphs, enantiomers, single isomer, or mixtures thereof. Pharmaceutically acceptable salts of pseudoephedrine include, without limitation thereto the hydrochloride and sulphate salts.

In another aspect, pseudoephedrine-containing particles are coated with a release-controlling polymer for modified release, whereas fexofenadine is provided for immediate release.

In one of the embodiments, pseudoephedrine or its pharmaceutically acceptable salt, optionally with a binder or adherent, is coated or deposited or layered or applied onto inert cores, which are further coated with a release-controlling polymer.

In one of the embodiments of the present invention, coating of a pseudoephedrine-containing solution or dispersion onto inert cores can be achieved by techniques including dipping, spraying, layering and the like.

In another aspect of the present invention, the pseudoephedrine coating is achieved by spraying using fluidized bed technology with Wurster, top spray or side spray techniques.

In another embodiment of the present invention, a pseudoephedrine-containing solution or dispersion is applied onto inert cores using a Wurster technology, wherein pseudoephedrine forms more than about 75% w/w, or more than about 60% w/w, of the pseudoephedrine-coated core.

In accordance with the invention, inert cores comprise pharmaceutically acceptable excipients, pellets, beads, spheres, particles or seeds that may be water-soluble, water swellable, or water-insoluble; and organic or inorganic, or mixtures thereof. The size of cores generally ranges from about 50 to about 5000 μm, or from about 100 to about 500 μm, or from about 150 to about 300 μm.

In the context of the present invention, pharmaceutically acceptable excipients serving as inert cores comprise insoluble pharmacologically inert materials, such as glass particles/beads or silicon dioxide, calcium phosphate dihydrate, dicalcium phosphate, calcium sulfate dihydrate, microcrystalline cellulose (e.g., Avicel™ supplied by FMC Corporation), silicified microcrystalline cellulose (e.g., Prosolv™ supplied by JRS Pharma), cellulose derivatives, powdered cellulose (e.g., Elcema™ G 250 manufactured by Degussa), or soluble cores such as sugar spheres having sugars like dextrose, lactose, anhydrous lactose, spray-dried lactose, lactose monohydrate, mannitol, starches, sorbitol, or sucrose, insoluble inert plastic materials such as spherical or nearly spherical core beads of polyvinyl chloride, polystyrene or any other pharmaceutically acceptable insoluble synthetic polymeric material, and the like and mixtures thereof.

In embodiments of the present invention, the inert cores comprise microcrystalline cellulose spheres such as CELPHERE® from Asahi Kasei Corporation, Tokyo, Japan, available in various grades. The properties of various commercially available CELPHERE products are summarized below:

| Parameter | SCP-100 | CP-102 | CP-203 | CP-305 | CP-507 | CP-708 |
|---|---|---|---|---|---|---|
| Particle size range (μm) | 75-212 | 106-212 | 150-300 | 300-500 | 500-710 | 710-850 |
| Sphericity | 1.2 | 1.2 | 1.1 | 1.1 | 1.2 | 1.2 |
| Bulk density (g/cm³) | 0.66 | 0.83 | 0.87 | 0.91 | 0.93 | 0.93 |
| Friability (%) | 0.0 | 0.0 | 0.0 | 0.0 | 0.0 | 0.0 |
| Water absorption (%) | 130 | 100 | 100 | 110 | 70 | 65 |

In embodiments of the present invention, a release-controlling polymer that provides a modified release of pseudoephedrine or a salt thereof can be water soluble, water swellable, water insoluble, pH dependent, pH independent or mixtures thereof.

Typical non-limiting examples of water soluble release-controlling polymers include cellulose derivatives such as carboxymethyl cellulose sodium, hydroxyethyl cellulose, hydroxypropyl methylcellulose (HPMC), homopolymers or copolymers of N-vinylpyrrolidone, vinyl and acrylic polymers, polyacrylic acid and the like. Non limiting examples of water insoluble release-controlling polymers include cellulose derivatives like ethyl cellulose, low substituted hydroxypropyl cellulose (L-HPC), cellulose acetate, cellulose propionate (lower, medium or higher molecular weight), cellulose acetate propionate, cellulose acetate butyrate, cellulose acetate phthalate, polyalkyl methacrylates, polyalkyl acrylates, polyvinyl acetate (PVA), chitosan, stearic acid, gum arabic, crosslinked vinylpyrrolidone polymers, hydrogenated castor oil, and the like. Other classes of release-controlling polymers or their mixtures in various ratios as required are also within the purview of this invention without limitation.

In one embodiment of the present invention, cores providing modified release of the decongestant, optionally with pharmaceutically acceptable excipients like diluents or fillers, are further granulated with a solution or dispersion comprising an antihistamine for immediate release.

In another embodiment, granulation of cores providing modified release of the decongestant, optionally with pharmaceutically acceptable excipients like diluents or fillers, is carried out in a rapid mixer granulator (RMG) or fluidized bed processor (FBP), such as with a top spray technique.

In one aspect of the present invention, the granulated blend comprising multi-particulate systems comprising modified release pseudoephedrine and fexofenadine are compressed into tablets without substantially damaging the modified release coating by use of pharmaceutical excipients that provide required cushioning during compression, or alternatively the systems are filled into capsules.

In another aspect of the present invention, the compressed tablets or capsules comprising pseudoephedrine for modified release and fexofenadine for immediate release are optionally further coated with a film coating and subsequently polished for aesthetic purposes by techniques known to a person skilled in the art.

In a further aspect of the present invention, the weight of a finished pharmaceutical formulation ranges between about 600 mg and about 1400 mg, or about 800 mg and about 1200 mg, per dosage unit.

In an embodiment the invention includes pharmaceutical formulations comprising fixed dose compositions of fexofenadine hydrochloride and pseudoephedrine hydrochloride producing plasma Tₘₐₓ values about 2 hours to about 4 hours for fexofenadine, and about 7 hours to about 11 hours for pseudoephedrine, after administration of a single dose containing 180 mg fexofenadine hydrochloride and 240 mg of pseudoephedrine hydrochloride to healthy humans.

In an embodiment the invention includes pharmaceutical formulations comprising fixed dose compositions of fexofenadine hydrochloride and pseudoephedrine hydrochloride producing plasma Cₘₐₓ values of fexofenadine ranging from about 315 ng/mL to about 473 ng/mL after administration of a single dose containing 180 mg fexofenadine hydrochloride and 240 mg of pseudoephedrine hydrochloride to healthy humans.

In an embodiment the invention includes pharmaceutical formulations comprising fixed dose compositions of fexofenadine hydrochloride and pseudoephedrine hydrochloride producing plasma AUC₀₋ₜ values of fexofenadine ranging from about 2022 ng·hour/ml to about 3034 ng·hour/ml after administration of a single dose containing 180 mg fexofenadine hydrochloride and 240 mg of pseudoephedrine hydrochloride to healthy humans.

In an embodiment the invention includes pharmaceutical formulations comprising fixed dose compositions of fexofenadine hydrochloride and pseudoephedrine hydrochloride producing plasma Cₘₐₓ values of pseudoephedrine ranging from about 289 ng/mL to about 415 ng/mL after administration of a single dose containing 180 mg fexofenadine hydrochloride and 240 mg of pseudoephedrine hydrochloride to healthy humans.

In an embodiment the invention includes pharmaceutical formulations comprising fixed dose compositions of fexofenadine hydrochloride and pseudoephedrine hydrochloride producing plasma AUC₀₋ₜ values of pseudoephedrine ranging from about 5188 ng·hour/mL to about 8784 ng·hour/mL after administration of a single dose comprising 180 mg fexofenadine hydrochloride and 240 mg of pseudoephedrine hydrochloride to healthy humans.

In the context of the present invention, during the processing of the pharmaceutical compositions into finished dosage formulations, one or more pharmaceutically acceptable excipients may optionally be used which include but are not limited to: diluents such as microcrystalline cellulose (MCC), silicified MCC (e.g. Prosolv™), powdered cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders or adherents such as acacia, guar gum, alginic acid, dextrin, maltodextrin, methylcellulose, ethyl cellulose, hydroxyethyl cellulose, hydroxypropyl cellulose (e.g., KLUCEL®), hydroxypropyl methylcellulose (e.g., METHOCEL®), carboxymethyl cellulose sodium, povidone (various grades of KOLLIDON® and PLASDONE®), starch and the like; disintegrants such as carboxymethyl cellulose sodium (e.g., Ac-Di-Sol®, Primellose®), crospovidone (e.g., Kollidon®, Polyplasdone®), povidone K-30, polacrilin potassium, starch, pregelatinized starch, sodium starch glycolate (e.g., Explotab®), and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like. Solvents that can be used in processing steps such as granulation, layering and coating include water methanol, ethanol, isopropyl alcohol, acetone, methylene chloride, dichloromethane, and the like and mixtures thereof.

Pharmaceutical compositions of the present invention may further include any one or more of pharmaceutically acceptable glidants, lubricants like sodium stearyl fumarate, opacifiers, colorants and other commonly used excipients.

The following examples illustrate certain specific aspects and embodiments of the invention and demonstrate the practice and advantages thereof. It is to be understood that the examples are given by way of illustration only and are not intended to limit the scope of the invention in any manner.

### EXAMPLE 1

### Fexofenadine Hydrochloride 180 mg Immediate Release and Pseudoephedrine Hydrochloride 240 mg Modified Release Tablets

| | |
|---|---|
| Ingredient | Kg |

DRUG COATING

| | |
|---|---|
| Microcrystalline cellulose spheres | 0.1 |
| (Celphere CP-102) | |
| Pseudoephedrine hydrochloride | 1.2 |
| Water‡ | 0.8 |

MODIFIED RELEASE COATING

| | |
|---|---|
| Ethyl Cellulose, 100 cP | 1^{$} |
| Acetyltributyl citrate | 0.3^{$} |
| Methylene chloride‡ | 41^{$} |

GRANULATION

| | |
|---|---|
| Fexofenadine hydrochloride | 0.9 |
| Silicified microcrystalline cellulose (Prosolv | 1.2 |
| HD 90)* | |
| Copovidone (Plasdone S-630)# | 0.2 |
| Isopropyl alcohol‡ | 4.4 |

LUBRICATION

| | |
|---|---|
| Silicified microcrystalline cellulose (Prosolv | 0.15 |
| HD 90) | |
| Croscarmellose sodium | 0.25 |
| Sodium stearyl fumarate | 0.03 |

FILM COATING

| | |
|---|---|
| Hydroxypropyl methyl cellulose, 5 cP | 0.09 |
| Polyethylene glycol 6000 | 0.15 |
| Talc | 0.02 |
| Titanium dioxide | 0.09 |
| Isopropyl alcohol‡ | 3.5 |
| Methylene chloride‡ | 3.5 |

POLISHING

| | |
|---|---|
| Hydrogenated vegetable oil (Type-I) | 0.1 |

‡Evaporates during processing.
*Prosolv HD 90 is manufactured by JRS Pharma GmbH Co. KG, Rosenberg, Germany.
#Plasdone S-630 is manufactured by International Specialty Products (ISP) Inc.

Manufacturing Process:

A. Drug Coating:
1. Pseudoephedrine hydrochloride was dissolved in water with stirring.
2. Solution of step 1 was coated onto microcrystalline cellulose spheres until the desired dose of the drug was built up using a fluidized bed coater (FBC) with a Wurster technique.

B. Modified Release Coating
3. Ethyl cellulose and acetyltributyl citrate were dispersed in methylene chloride with stirring to get a uniform dispersion.
4. Drug-coated microcrystalline cellulose spheres of step 2 were further coated with the dispersion of step 3 using a fluidized bed coater (FBC) with a Wurster technique until the desired weight build-up was obtained.
5. The coated particles of step 4 were dried in the FBC at 60±5° C. for 2 hours.

C. Granulation
6. Fexofenadine hydrochloride and copovidone were dispersed in isopropyl alcohol with stirring.
7. Dried particles of step 5 were mixed with silicified microcrystalline cellulose, and granulated with the dispersion of step 6 in a fluidized bed processor using top-spray technique.
8. The granules of step 7 were dried at 55±5° C. until the loss on drying (LOD) was less than 2% w/w as determined at 105° C.

D. Lubrication
9. Dried granules of step 8 were lubricated by mixing with silicified microcrystalline cellulose, croscarmellose sodium and sodium stearyl fumarate in a double cone blender.

E. Compression
10. Lubricated blend of step 9 was compressed into 5,000 tablets using a 21×10 mm punch set in a rotary compression machine to get an average tablet weight of 1000 mg and average tablet hardness in the range of 8-16 kP (kilopond).

F. Film Coating
11. Hydroxypropyl methylcellulose, polyethylene glycol, talc and titanium dioxide were dispersed in the mixture of isopropyl alcohol and methylene chloride with stirring to get a uniform dispersion.
12. Tablets of step 10 were coated with the dispersion of step 11 using a pan coating technique until the desired weight build-up was obtained.

G. Polishing
13.Coated tablets of step 12 were polished with hydrogenated vegetable oil using a pan coater by sprinkling vegetable oil over the warmed bed of tablets and tumbling for 30-45 minutes.

In vitro release profile of the product of Example 1 in comparison with a commercial product was determined with the following parameters:
Media: 0.001 N HCl.
Volume: 900 ml.
Apparatus: USP apparatus 2 (Paddle) from Test 711 “Dissolution” in United States Pharmacopeia 24, United States Pharmacopeial Convention, Inc., Rockville, Md. (1999).

Speed: 50 rpm.

| | Cumulative % Drug Dissolved | | | |
|---|---|---|---|---|
| | Fexofenadine | | Pseudoephedrine | |
| Time | | ALLEGRA-D ® | | ALLEGRA-D ® |
| (Hours) | Example 1 | 24 HOUR | Example 1 | 24 HOUR |
| 0.5 | 93 | 88 | — | — |
| 1 | 96 | 89 | 10 | 2 |
| 5 | — | — | 49 | 27 |
| 11 | — | — | 76 | 72 |
| 19 | — | — | 91 | 88 |
| 23 | — | — | 94 | 93 |

### EXAMPLE 2

### Fexofenadine Hydrochloride 180 mg and Pseudoephedrine Hydrochloride 240 mg Modified Release Tablets

| | |
|---|---|
| Ingredient | Kg |

DRUG COATING

| | |
|---|---|
| Microcrystalline cellulose spheres | 0.1 |
| (Celphere CP-102) | |
| Pseudoephedrine hydrochloride | 1.26 |
| Water‡ | 0.84 |

MODIFIED RELEASE COATING

| | |
|---|---|
| Ethyl cellulose, 100 cps | 1.01 |
| Acetyltributyl citrate | 0.25 |
| Methylene chloride‡ | 10.96 |

GRANULATION

| | |
|---|---|
| Fexofenadine hydrochloride | 0.95 |
| Silicified microcrystalline cellulose | 0.75 |
| (Prosolv HD 90)** | |
| Copovidone (Plasdone S-630) | 0.11 |
| Polyethylene glycol 400 | 0.11 |
| Croscarmellose sodium NF | 0.26 |
| Isopropyl alcohol‡ | 5.67 |

LUBRICATION

| | |
|---|---|
| Silicified microcrystalline cellulose | 0.6 |
| (Prosolv HD 90) | |
| Sodium stearyl fumarate | 0.03 |

FILM COATING

| | |
|---|---|
| Hydroxypropyl methylcellulose, 5 cps | 0.11 |
| Polyethylene glycol 6000 | 0.19 |
| Talc | 0.01 |
| Titanium dioxide | 0.11 |
| Isopropyl alcohol‡ | 5.34 |
| Methylene chloride‡ | 2.66 |

POLISHING

| | |
|---|---|
| Hydrogenated vegetable oil (Type I) | 0.02 |

‡Evaporates during processing.

Manufacturing process: same as that of Example 1.

In vitro release profile of product of Example 2 in comparison with a marketed product was determined with the following parameters:
Media: 0.1 N HCl, pH 4.5 acetate buffer, pH 6.8 phosphate buffer.
Volume: 900 ml.
Apparatus: USP apparatus II (Paddle).
Speed: 50 rpm.

The dissolution profile of fexofenadine in the product was compared with that of the commercial product ALLEGRA-D® 24 Hour and the data are reported below.

| | Cumulative % Fexofenadine Dissolved | | | | | |
|---|---|---|---|---|---|---|
| | ALLEGRA- | | ALLEGRA- | | ALLEGRA- | |
| | D ® 24 | | D ® 24 | | D ® | |
| | HOUR | Example 2 | HOUR | Example 2 | 24 HOUR | Example 2 |
| Time | 0.1N | | pH 4.5 | | pH 6.8 Phosphate | |
| (minutes) | HCl | | Acetate Buffer | | Buffer | |
| 10 | 68 | 66 | 62 | 76 | 75 | 85 |
| 20 | 85 | 75 | 75 | 93 | 88 | 101 |
| 30 | 89 | 80 | 77 | 97 | 92 | 105 |
| 45 | 92 | 86 | 78 | 99 | 95 | 107 |

The dissolution profile of pseudoephedrine in the product was compared with that of the commercial product ALLEGRA-D® 24 Hour and the data are reported below.

| | Cumulative % Pseudoephedrine Dissolved | | | | | |
|---|---|---|---|---|---|---|
| | ALLEGRA- | | ALLEGRA- | | ALLEGRA- | |
| | D ® 24 | | D ® 24 | | D ® | |
| | HOUR | Example 2 | HOUR | Example 2 | 24 HOUR | Example 2 |
| Time | 0.1N | | pH 4.5 | | pH 6.8 Phosphate | |
| (minutes) | HCl | | Acetate Buffer | | Buffer | |
| 1 | 2 | 2 | 1 | 1 | 2 | 2 |
| 3 | 18 | 17 | 15 | 16 | 16 | 17 |
| 7 | 47 | 46 | 43 | 46 | 46 | 47 |
| 11 | 72 | 66 | 68 | 66 | 69 | 68 |
| 23 | 98 | 91 | 93 | 94 | 95 | 89 |

### EXAMPLE 3

### Stability Studies for Compositions Prepared According to Example 2

Tablets prepared according to Example 2 were packaged in two different packages and exposed to accelerated stability conditions (40° C./75% RH) for three months, and samples were analyzed at intervals for related impurities, dissolution and purity.
Example 3a: 30 tablets were packed in closed 85 ml HDPE containers with desiccant (a combination of silica gel and charcoal).
   Example 3b: 10 tablets were packed in clear blisters made of blister forming material as 250 μm PVC laminated with 23 μm Aclar and sealed with lidding material 25-30 μm hard tempered aluminum foil.

The data from tested parameters are given below in Table A (fexofenadine) and Table B (pseudoephedrine).

TABLE A

| | Example 3a | | | | Example 3b | | | |
|---|---|---|---|---|---|---|---|---|
| | | 1 | 2 | 3 | | 1 | 2 | 3 |
| Parameter | Initial | Mo. | Mo. | Mo. | Initial | Mo. | Mo. | Mo. |
| Purity (%) | 99.4 | 97.9 | 100.4 | 97.5 | 98.2 | 98.8 | 101.4 | 97.5 |
| Compound A (%) | 0.07 | 0.08 | 0.09 | 0.1 | 0.08 | 0.08 | 0.09 | 0.09 |
| Unknown (%) | 0.05 | 0.04 | 0.04 | 0.05 | 0.04 | 0.04 | 0.05 | 0.04 |
| Total Impurities | 0.15 | 0.19 | 0.25 | 0.25 | 0.2 | 0.19 | 0.26 | 0.23 |
| Fexofenadine | 93.5 | 96.6 | 99.5 | 99.5 | 93.5 | 95.1 | 100 | 101 |
| dissolved in 900 ml | | | | | | | | |
| 0.001 N HCl in 30 | | | | | | | | |
| minutes (%) | | | | | | | | |

TABLE B

| | | Example 3a | | | | Example 3b | | | |
|---|---|---|---|---|---|---|---|---|---|
| Parameter | | Initial | 1 Mo. | 2 Mo. | 3 Mo. | Initial | 1 Mo. | 2 Mo. | 3 Mo. |
| Purity (%) | | 102 | 101 | 100 | 101 | 100 | 99 | 99 | 101 |
| Unknown (%) | | NA | 0.006 | 0.006 | 0.006 | 0.003 | 0.007 | 0.07 | 0.005 |
| Total Impurities | | NA | 0.02 | 0.01 | 0.006 | 0.01 | 0.02 | 0.07 | 0.006 |
| Cum. % | 3 | 19 | 16 | 17 | 17 | 19 | 18 | 20 | 20 |
| Pseudoephedrine | hours | | | | | | | | |
| Dissolved | 7 | 52 | 46 | 47 | 47 | 52 | 47 | 52 | 52 |
| | hours | | | | | | | | |
| | 23 | 95 | 89 | 92 | 91 | 95 | 87 | 92 | 92 |
| | hours | | | | | | | | |

### EXAMPLE 4

### Pharmacokinetic Parameters of Formulations Prepared According to Example 2.

Tablets were evaluated in a randomized single dose crossover bioequivalence study involving administration of the test product and the commercial product ALLEGRA-D 24 Hour to 40 (fed) and 62 (fasting) healthy human volunteers, and plasma concentrations were determined at intervals after dosing.

The following parameters were calculated:

AUCO₀₋ₜ=the area under plasma concentration versus time curve, from time zero to the last measurable concentration.

AUC_{0-∞}=area under the plasma concentration versus time curve, from time zero to infinity.

Cₘₐₓ=maximum plasma concentration.

Tₘₐₓ=Time of the maximum measured plasma concentrations.

The results of these pharmacokinetic parameters in the fasting study were calculated and are summarized in Table C and pharmacokinetic parameters in the fed study were calculated and are summarized in Table D.

TABLE C

| | | | | |
|---|---|---|---|---|
| | | ALLEGRA-D ® | | |
| | Example 2 | 24 HOUR | Ratio | 90% C.I. |
| Parameters | (T) | (R) | (T/R) | (%) |

Fexofenadine

| | | | | |
|---|---|---|---|---|
| AUC₀₋ₜ | 4772 | 4869 | 98.02 | 89.86-106.9 |
| (ng · hour/mL) | | | | |
| AUC_{0-∞} | 4824 | 4929 | 97.88 | 89.8-106.6 |
| (ng · hour/mL) | | | | |
| Cₘₐₓ (ng/mL) | 768 | 773 | 99.3 | 90.87-108.5 |
| Tₘₐₓ (hours) | 1.67 | 1.51 | — | — |

Pseudoephedrine

| | | | | |
|---|---|---|---|---|
| AUC₀₋ₜ | 5775 | 6359 | 93.73 | 83.06-99.31 |
| (ng · hour/mL) | | | | |
| AUC_{0-∞} | 6284 | 6765 | 90.82 | 86.39-99.87 |
| (ng · hour/mL) | | | | |
| Cₘₐₓ (ng/mL) | 315 | 336 | 92.88 | 88.81-98.92 |
| Tₘₐₓ (hours) | 9.5 | 10 | — | — |

TABLE D

| | | | | |
|---|---|---|---|---|
| | | ALLEGRA-D ® | | |
| Parameters | Example 2 | 24 HOUR | Ratio | 90% C.I. (%) |

Fexofenadine

| | | | | |
|---|---|---|---|---|
| AUC₀₋ₜ (ng · | 2528 | 2389 | 105.8 | 100.2-111.8 |
| hour/mL) | | | | |
| AUC_{0-∞} (ng · | 2585 | 2444 | 105.7 | 100.3-111.5 |
| hour/mL) | | | | |
| Cₘₐₓ (ng/mL) | 394 | 434 | 90.6 | 82.56-99.4 |
| Tₘₐₓ (hours) | 3 | 2.25 | — | — |

Pseudoephedrine

| | | | | |
|---|---|---|---|---|
| AUC₀₋ₜ (ng · | 6486 | 6345 | 102.2 | 96.3-108.3 |
| hour/mL) | | | | |
| AUC_{0-∞} (ng · | 6570 | 6453 | 101.8 | 96.0-107.9 |
| hour/mL) | | | | |
| Cₘₐₓ (ng/mL) | 362 | 346 | 104.7 | 99.5-110.0 |
| Tₘₐₓ (hour) | 9 | 12.5 | — | — |

### EXAMPLE 5

### Fexofenadine Hydrochloride 180 mg and Pseudoephedrine Hydrochloride 240 mg Modified Release Capsules

| | |
|---|---|
| Ingredient | Quantity/Batch |

SEAL COATING

| | |
|---|---|
| Dicalcium phosphate | 85 |
| Ethyl cellulose 10 cps | 10.34 |
| Hydroxypropyl methylcellulose 5 cps | 2.55 |
| Acetlytributyl citrate | 2.86 |
| Isopropyl alcohol‡ | 130.17 |
| Methylene chloride‡ | 130.17 |
| Water‡ | 38.9 |

DRUG LOADING

| | |
|---|---|
| Pseudoephedrine hydrochloride | 1260 |
| Water‡ | 840 |

MODIFIED RELEASE COATING

| | |
|---|---|
| Ethyl cellulose 100 cps | 756 |
| Acetyltributyl citrate | 189 |
| Methylene chloride‡ | 22680 |

LAYERING

| | |
|---|---|
| Fexofenadine hydrochloride | 945 |
| Croscaramellose sodium | 94.5 |
| Mannitol | 378 |
| Microcrystalline cellulose (Avicel PH105)# | 383.25 |
| Talc | 36.75 |
| Isopropyl alcohol‡ | 7350 |

‡Evaporates during processing.
#Avicel PH105 is supplied by FMC Corporation.

Manufacturing Process:

A. Seal Coating:
1. Ethyl cellulose, hydroxypropyl methylcellulose and acetlytributyl citrate were dispersed in methylene chloride under stirring to get a uniform dispersion.
2. Isopropyl alcohol was added to step 1 with stirring until a clear solution was formed and to the clear solution water was added with stirring.
3. Solution of step 2 was coated onto dicalcium phosphate particles which passed through a ASTM #80 mesh sieve and were retained on a ASTM # 100 mesh sieve.

B. Drug Loading:
4. Pseudoephedrine hydrochloride was dissolved in water with stirring.
5. Solution of step 4 was coated onto seal coated dicalcium phosphate using a fluidized bed coater (FBC) having an inlet temperature in the range of 30-50° C. and product temperature of 25-40° C., using a Wurster technique, until the desired weight build-up was obtained. Dried the drug coated pellets until loss on drying at 105° C. was not more than 2% w/w. The pseudoephedrine loaded pellets were passed through a ASTM #35 mesh sieve and retained on a ASTM # 50 mesh sieve.

C. Modified Release Coating
6. Ethyl cellulose and acetyltributyl citrate were dissolved in methylene chloride under constant stirring to get a uniform dispersion.
7. Drug coated pellets of step 5 were further coated with the dispersion of step 6 using a fluidized bed coater (FBC) with inlet air temperature of 30-50° C. and product temperature of 25-40° C., using a Wurster technique, until desired weight build-up was obtained. Dried the coated pellets until loss on drying at 105° C. was not more than 2% w/w. The pseudoephedrine modified release pellets were passed through a ASTM #30 mesh sieve and retained on a ASTM # 40 mesh sieve.

D. Drug Loading
8. Fexofenadine hydrochloride, croscarmellose, mannitol, microcrystallilne cellulose and talc were dispersed in isopropyl alcohol with stirring.
9. Modified release coated pellets of step 7 were further coated with dispersion of step 8 using a fluidized bed coater (FBC) with inlet air temperature of 30-50° C. and product temperature of 25-40° C., using a Wurster technique, until the desired weight build-up was obtained.

G. Capsule Filling
10. Pellets of step 9 were filled into hard gelatin capsules.

### EXAMPLE 6

### Fexofenadine Hydrochloride Layered over Pseudoephedrine Hydrochloride Modified Release Pellets, and then Compressed into Tablets

| | |
|---|---|
| Ingredient | Kg |

SEAL COATING

| | |
|---|---|
| Dicalcium phosphate, as in Example 5 | 85 |
| Ethyl cellulose 10 cps | 10.34 |
| Hydroxypropyl methylcellulose 5 cps | 2.55 |
| Acetylbutyl citrate | 2.86 |
| Isopropyl alcohol‡ | 130.17 |
| Methylene chloride‡ | 130.17 |
| Water‡ | 38.90 |

DRUG LOADING

| | |
|---|---|
| Pseudoephedrine hydrochloride | 1260 |
| Water‡ | 840 |

MODIFIED RELEASE COATING

| | |
|---|---|
| Ethyl cellulose 100 cps | 756 |
| Acetyltributyl citrate | 189 |
| Methylene chloride‡ | 22680 |

LAYERING

| | |
|---|---|
| Fexofenadine hydrochloride | 945 |
| Croscarmellose sodium | 94.5 |
| Mannitol | 378 |
| Microcrystalline cellulose | 383.25 |
| (Avicel PH105) | |
| Talc | 36.75 |
| Isopropyl alcohol‡ | 7350 |

BLENDING

| | |
|---|---|
| Powdered cellulose | 1250 |
| Croscarmellose sodium | 125 |
| Sodium stearyl fumarate | 25 |

‡Evaporates during processing.

Manufacturing process: same as above Example 5 for steps A-D.

E. Blending:
Fexofenadine hydrochloride layered pseudoephedrine modified release pellets were mixed with powdered cellulose, croscarmellose sodium and sodium stearyl fumarate in a double cone blender at 20 rpm to get a lubricated blend.

F. Compression:
Lubricated blend from the above step was compressed into 5,000 tablets using 21.6 mm round punch set in a rotary compression machine to get an average tablet weight of 1000 mg.

G. Coating & Polishing:
Tablets from above can be further coated and polished with hydrogenated vegetable oil using a pan coater, by sprinkling vegetable oil over a warmed bed of tablets and tumbling for 30-45 minutes.

The fexofenadine and pseudoephedrine salts used in all of the above examples had the following physical parameters:

| | Fexofenadine | Pseudoephedrine |
|---|---|---|
| Parameter | Hydrochloride | Hydrochloride |
| Bulk density | 0.12 | 0.52 |
| (g/ml) | | |
| Tap density | 0.25 | 0.65 |
| (g/ml) | | |
| Compressibillty | 52 | 19 |
| Index | | |
| Hausner Ratio | 0.48 | 0.8 |

Particle Size Distribution

| | |
|---|---|
| | Psuedoephedrine HCl |
| | Measured by |
| Fexofenadine HCl | Sieve Analysis |

| Measured by Malvern | Aperture | % Passing |
|---|---|---|
| Mastersizer | Size (μm) | Through |
| D₉₀ 38 μm | 500 | 0.1 |
| D₅₀ 9 μm | 250 | 33.7 |
| D₁₀ 2 μm | 150 | 68.7 |

1. A pharmaceutical composition comprising a plurality of formulated particles providing a modified release of pseudoephedrine or a salt thereof into aqueous fluids, granulated with a granulating composition containing fexofenadine or a salt thereof.

2. The pharmaceutical composition of claim 1, wherein formulated particles comprise pharmaceutically inert particulate cores having a coating comprising pseudoephedrine or a salt thereof.

3. The pharmaceutical composition of claim 1, wherein formulated particles comprise cellulose cores.

4. The pharmaceutical composition of claims 1, wherein formulated particles are provided with an outer polymer coating.

5. The pharmaceutical composition of claim 1, wherein formulated particles are provided with an outer polymer coating that modifies release of pseudoephedrine or a salt thereof.

6. The pharmaceutical composition of claim 1, wherein an outer polymer coating comprises ethyl cellulose.

7. The pharmaceutical composition of claim 1, wherein pseudoephedrine is present as pseudoephedrine hydrochloride.

8. The pharmaceutical composition of claim 1, wherein fexofenadine is present as fexofenadine hydrochloride.

9. The pharmaceutical composition of claim 1, optionally together with one or more pharmaceutical excipients, compressed into a tablet.

10. The pharmaceutical composition of claim 1, producing pseudoephedrine Cₘₐₓ values about 289 ng/mL to about 415 ng/mL and AUC₀₋ₜ values about 5188 ng·hour/mL to about 8784 ng·hour/mL, after oral administration of a single dose containing 240 mg of a pseudoephedrine salt to healthy humans.

11. The pharmaceutical composition of claim 1, producing fexofenadine Cₘₐₓ values about 315 ng/mL to about 473 ng/mL and AUC₀₋ₜ values about 2022 ng·hour/mL to about 3034 ng·hour/mL, after oral administration of a single dose containing 180 mg of a fexofenadine salt to healthy humans.

12. The pharmaceutical composition of claim 1, producing Tₘₐₓ values about 2 hours to about 4 hours for fexofenadine, and about 7 hours to about 11 hours for pseudoephedrine, after oral administration of a single dose to healthy humans.

13. A pharmaceutical composition comprising a plurality of cellulose particles having a coating comprising pseudoephedrine or a salt thereof and having an outer coating comprising a polymer and providing a modified release of pseudoephedrine or a salt thereof into aqueous fluids, granulated with a granulating composition containing fexofenadine or a salt thereof.

14. The pharmaceutical composition of claim 13, wherein cellulose particles are coated with a coating comprising pseudoephedrine hydrochloride.

15. The pharmaceutical composition of claim 13, wherein an outer coating comprises ethyl cellulose.

16. The pharmaceutical composition of claim 13, wherein coated cellulose particles are granulated with a composition comprising fexofenadine hydrochloride.

17. The pharmaceutical composition of claim 13, optionally together with one or more pharmaceutical excipients, compressed into a tablet.

18. The pharmaceutical composition of claim 13, producing pseudoephedrine Cₘₐₓ values about 289 ng/mL to about 415 ng/mL and AUC₀₋ₜ values about 5188 ng·hour/mL to about 8784 ng·hour/mL, after oral administration of a single dose containing 240 mg of a pseudoephedrine salt to healthy humans.

19. The pharmaceutical composition of claim 13, producing fexofenadine Cₘₐₓ values about 315 ng/mL to about 473 ng/mL and AUC₀₋ₜ values about 2022 ng·hour/mL to about 3034 ng·hour/mL, after oral administration of a single dose containing 180 mg of a fexofenadine salt to healthy humans.

20. The pharmaceutical composition of claim 13, producing Tₘₐₓ values about 2 hours to about 4 hours for fexofenadine, and about 7 hours to about 11 hours for pseudoephedrine, after oral administration of a single dose to healthy humans.

21. A pharmaceutical composition comprising:
a) a plurality of cellulose particles having a coating comprising pseudoephedrine hydrochloride, an outer coating comprising ethyl cellulose, and providing a modified release of pseudoephedrine or a salt thereof into aqueous fluids, granulated with a granulating composition containing fexofenadine hydrochloride; and
b) one or more pharmaceutical excipients; compressed into a tablet.
